(19) United States
(12) Patent Application Publication
(10) Pub. No.: US 2003/0130697 A1
(43) Pub. Date: Jul. 10, 2003

(54) System and/or method for refibrillation of the heart for treatment of post-countershock pulseless electrical activity and/or asystole

(76) Inventors: Henry R. Halperin, Baltimore, MD (US); Charles T. Leng, Timonium, MD (US); Ronald D. Berger, Baltimore, MD (US)

Correspondence Address: NIXON & VANDERHYE, PC, 1100 N GLEBE ROAD, 8TH FLOOR, ARLINGTON, VA, 22201-4714, US

(21) Appl. No.: 10/278,010

(22) Filed: Oct. 23, 2002

Related U.S. Application Data
(60) Provisional application No. 60/343,155, filed on Oct. 23, 2001.

(51) Int. Cl. A61N 1/39

(52) U.S. Cl. 607/2; 607/5; 607/3

(57) ABSTRACT

A method and/or system for inducing ventricular fibrillation (VF) of the heart for treatment of post-countershock pulseless electrical activity (PEA) or asystole. In certain example embodiments, it has been found that reinduction of ventricular fibrillation, followed by restoration of blood flow with cardiopulmonary resuscitation (CPR), can make subsequent countershocks more successful in restoring a heart rhythm associated with blood flow.

This application claims priority on U.S. Provisional Application No. 60/343,155, filed Oct. 23, 2001, the disclosure of which is hereby incorporated herein by reference.

This application relates to a method and/or system for re-inducing ventricular fibrillation (VF) of the heart for treatment of post-countershock pulseless electrical activity (PEA) and/or asystole. In certain example embodiments, reinduction of VF, followed by restoration of blood flow with cardiopulmonary resuscitation (CPR), can make subsequent countershocks more successful in restoring a heart rhythm associated with blood flow and/or can significantly increase a patient's chances of surviving PEA and/or asystole.

### BACKGROUND AND SUMMARY OF THE INVENTION

Many people die yearly from sudden cardiac death. In most of these cases, the cause of death is ventricular tachycardia and/or ventricular fibrillation (VF). Known treatments include the use of automatic implantable cardioverting/defibrillating devices, and automatic external defibrillating devices which have been used in attempts to prevent sudden cardiac death from these causes.

Cardioversion (performed by a cardioverter) may be defined as the correction of either ventricular tachycardia (VT) or ventricular fibrillation (VF) by the discharge of electrical energy (e.g., shock) into the heart. The shock may be either synchronized or non-synchronized. Ventricular fibrillation is generally an abnormally rapid heartbeat disorder, disorganized and irregular, or non-periodic, and is often fatal unless corrected within a number of minutes by the discharge of electrical energy through the heart. Defibrillation may be effected by non-synchronized delivery of electrical energy to the heart to correct ventricular fibrillation. A plurality of different types of implantable cardioverter defibrillation (ICD) systems are known in the art. For example, see each of U.S. Pat. Nos. 4,030,509; 4,662,377; 5,133,365; and 6,067,471; the disclosures of which are all hereby incorporated herein by reference. ICD systems may be used to provide electric shock to the heart in order to correct (i.e., terminate) VF. While certain ICDs are capable of inducing VF, this is only done in order to test the operation of the ICD whose purpose is to terminate VF. In addition, a number of automatic external defibrillation devices are also known (e.g., see U.S. Pat. Nos. 6,427,083, 6,356,785, 6,321,113, 6,263,238, and 6,246,907).

Thus, it can be seen that the conventional treatment for ventricular fibrillation (VF) comprises the use of electrical shocks or countershocks to the heart in order to terminate the fibrillation. Such shocks or countershocks to the heart have been found to be effective in terminating VF and helping the patient recover when the VF is of relatively short duration (i.e., when the VF lasts less than a few minutes).

Unfortunately, in the event of prolonged VF (e.g., VF lasting more than a few minutes), the use of conventional electrical shocks or countershocks has been found to be problematic. In particular, countershock termination of prolonged VF frequently results in either pulseless electrical activity (PEA) or asystole (both of which often lead to patient death). Patient resuscitation from these postcountershock rhythms rarely proves successful, with the short-term mortality rate reportedly being at least 85%. Accordingly, postcountershock PEA and asystole are often viewed as terminal rhythms.

Recurrent episodes of VF have conventionally been considered to be major setbacks to cardiac resuscitation, since prolonged VF often leads to death. Thus, it will be appreciated by those skilled in the art that recurrent VF has for years been thought to be highly problematic and undesirable.

Quite surprisingly, it has been found that re-induction of VF (i.e., refibrillation or RF) is highly beneficial when performed on a patient's heart that is suffering from pulseless electrical activity (PEA) and/or asystole as a result of initial defibrillation. This is especially the case after prolonged VF in the first place, as PEA and asystole most often occur after prolonged VF. After re-induction of VF (i.e., after RF), restoration of blood flow with cardiopulmonary resuscitation (CPR) can make subsequent countershocks more successful in restoring a normal heart rhythm associated with blood flow. It has been found that the use of RF in such a manner may enable the chance of survival from postcountershock PEA and/or asystole to be improved substantially when using conventional defibrillation together with subsequent RF in the event of PEA and/or asystole.

The use of RF to treat postcountershock PEA and/or asystole is a significant improvement over conventional practice. Moreover, the use of RF in such a manner flies directly in the face of conventional practice, since recurrent VF has for years been viewed as undesirable and likely to cause death.

In certain example embodiments of this invention, there is provided a method of treating PEA and/or asystole in a heart of a patient, the method comprising: determining if the heart is in PEA or asystole; and when it is determined that the heart is in PEA or asystole, then applying electric energy (e.g., shock) to the heart in order to induce VF in the heart and thereafter performing CPR.

In other example embodiments of this invention, there is provided a device for inducing VF in a heart of a patient, the device comprising: detection circuitry for determining whether the heart is in PEA or asystole; and a plurality of electrodes for inducing VF in the heart in response to detecting at least that the heart is in PEA or asystole.

### BRIEF DESCRIPTION OF THE DRAWINGS

FIG. 1 is a flowchart illustrating certain steps carried out in certain embodiments of this invention.

FIG. 2 is a schematic diagram of electrodes used to apply electric shock and/or energy to the heart in order to induce ventricular fibrillation (VF) according to an example embodiment of this invention.

FIG. 3 is a flowchart illustrating certain steps carried out according to another example embodiment of this invention.

FIG. 4 is a block diagram of an RF device which may be used in carrying out one or more of the steps illustrated in the FIG. 3 embodiment of this invention.

FIG. 5 is a schematic diagram of a device (e.g., from the embodiment of FIGS. 3-4) that may be used in order to induce VF in order to treat PEA or asystole in certain embodiments of this invention.

### DETAILED DESCRIPTION OF THE INVENTION

As explained above, countershock termination of prolonged ventricular fibrillation (VF) frequently results in pulseless electrical activity (PEA) and/or asystole, neither of which allow the heart to beat and produce blood flow. Resuscitation from these postcountershock rhythms (PEA and asystole) rarely proves successful given conventional techniques.

According to certain example embodiments of this invention, it has surprisingly been found that re-induction of ventricular fibrillation (VF) (i.e., refibrillation or RF), followed by restoration of blood flow with cardiopulmonary resuscitation (CPR), is highly beneficial when performed on a patient's heart suffering from pulseless electrical activity (PEA) and/or asystole. This has been found to make subsequent countershocks more successful in restoring a heart rhythm associated with good blood flow. As explained above, PEA and/or asystole often arise after electrical shock(s)/countershock(s) is/are used in order to terminate prolonged VF. It has unexpectedly been found that the use of RF in such a manner may enable the chance of survival from postcountershock PEA and/or asystole to be improved substantially.

An example RF device may monitor the heart beat, and if ventricular fibrillation (VF) is followed by PEA or asystole, and there is a measured lack of blood flow, then the device can deliver electrical energy to reinduce ventricular fibrillation. According to certain example embodiments of this invention, such a device may be incorporated into, or work alongside, a defibrillation device that is used in the treatment of cardiac arrest. Two or more electrodes may be attached to the chest, similar to the connections of standard defibrillators. The electrodes may be attached to an amplifier and processing system that monitors an electrocardiogram. Recognition algorithms that can detect VF and/or ventricular tachycardia, as well as the absence of these rhythms, may be provided. There also may be provided algorithms, stored in the device or accessible by the device, that can detect cardiac mechanical activity and blood flow from electrical signals of the body. If the device detects ventricular tachycardia and/or ventricular fibrillation (VF), then detects a defibrillation shock being delivered, then detects the absence of ventricular tachycardia, the absence of ventricular fibrillation, and the absence of blood flow, it may ask the operator if it is desired to reinduce ventricular fibrillation. If so, then the device may be activated by the operator so as to deliver electrical energy through the electrodes to reinduce ventricular fibrillation (i.e., perform refibrillation or RF).

For purposes of example only, a study was performed on animals (although the instant invention is clearly also relevant to and may be used on humans). In the study of defibrillation after prolonged VF, intentional refibrillation (RF) conferred several advantages to experimental animals randomized to the RF group. The additional cycle of electrical refibrillation and delayed defibrillation resulted in significantly shorter resuscitation times than for our control subjects despite identical administration of CPR, epinephrine, and immediate countershocks to profoundly ischemic myocardium after 12 minutes of unsupported VF. Intentional RF also allowed a simpler and more predictable resuscitation course, with immediate return of spontaneous circulation (ROSC) providing a consistent end point after delayed defibrillation at the 16-minute time point. However, possibly the most important finding of that study was that survival from postcountershock PEA and asystole could effectively be transformed in the experimental model from 1 of 5 (20%) (immediate defibrillation group) to 5 of 5 (RF group) through the intentional use of electrical refibrillation (RF). These findings fly in the face of and are directly opposite the conventional widely held perception of postcountershock PEA and asystole as terminal rhythms and suggest that electrical RF may prove capable of favorably modifying the normally terminal prognosis of these rhythms.

According to certain example embodiments of this invention, it may be desirable to perform intentional RF only when the heart (of a human or animal) is not generating blood flow. Thus, a safety feature in certain example non-limiting embodiments of this invention may be used to prevent unnecessary or accidental re-fibrillation as follows. The safety system determines whether the heart is generating satisfactory blood flow before delivering energy to fibrillate (e.g., refibrillate) the heart. If the heart is beating and generating satisfactory blood flow, the system will not allow delivery of energy. One example way of determining whether the heart is beating and generating satisfactory flow is to measure transthoracic impedance through the same electrodes applied to the body to deliver the fibrillation energy. A high frequency electric signal may be applied between the electrodes, and an impedance circuit can measure the impedance of the body tissue that the signal is traversing. This impedance may be determined by measuring the amount of current and/or the amount of voltage flowing through the tissue as a result of the application of the high frequency signal, and then using known processing techniques to determine the impedance. An example processing technique in this regard is to divide the magnitude of the voltage by the magnitude of the current. If the heart is beating, the impedance will change in synchrony with the heart beat.

Thus, according to this example safety feature, if impedance signals which are characteristic of satisfactory beating hearts are measured, the system will not deliver fibrillation (e.g., RF) energy. If impedance signals not characteristic of satisfactory beating hearts are measured, the system will deliver fibrillation energy when activated by the operator. This method is similar to the operation of automatic defibrillators, where the defibrillator analyzes the electrocardiogram and will allow delivery of defibrillation shocks only if certain cardiac rhythms are present (ventricular tachycardia or ventricular fibrillation). There are of course other ways to determine if the heart is beating, but the basic principal is to determine if the heart is beating in a satisfactory manner. For instance, one could use imaging techniques, and/or apply microwaves, ultrasound, and/or near infrared radiation to determine if there is satisfactory blood flow (i.e., to determine if the heart is beating in a satisfactory manner) in other example embodiments of this invention.

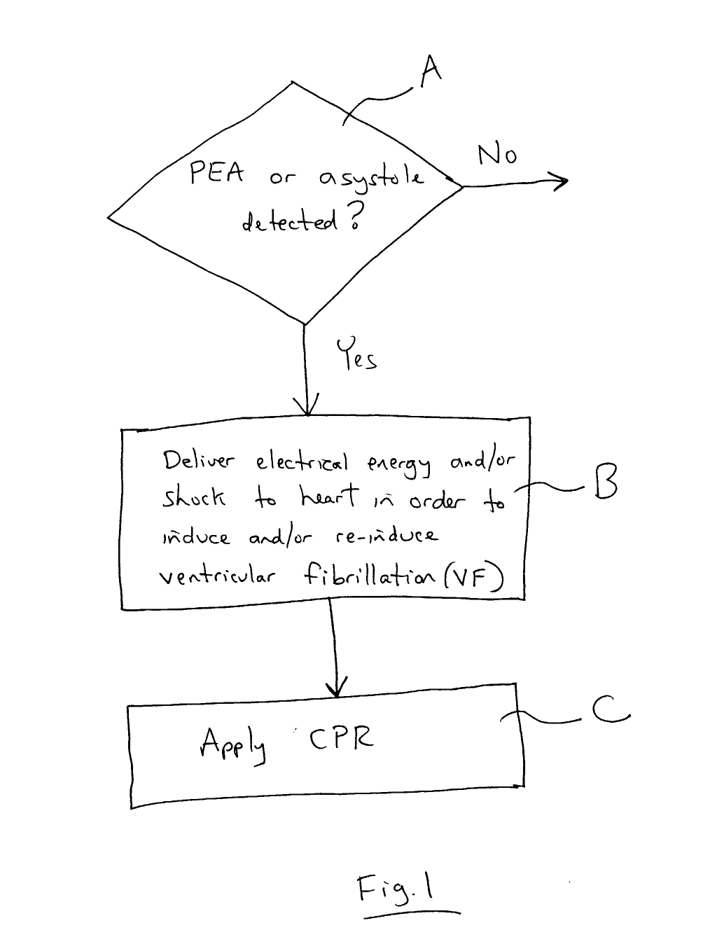

FIG. 1 illustrates steps carried out in accordance with an example embodiment of this invention. First, it is determined whether the patient's heart is suffering from PEA and/or asystole (step A). If so, then electrodes may be used to deliver electric energy such as shock (the term “shock” as used herein includes countershock) to the heart in a manner so as to induce ventricular fibrillation (VF) (step B). In certain instances, such an induction of VF may be referred to as refibrillation (RF) if the heart has previously experienced VF and the PEA and/or asystole was caused by termination of the initial VF. After the VF as been induced and/or re-induced, CPR is applied to the heart in order to restore bloodflow (step C).

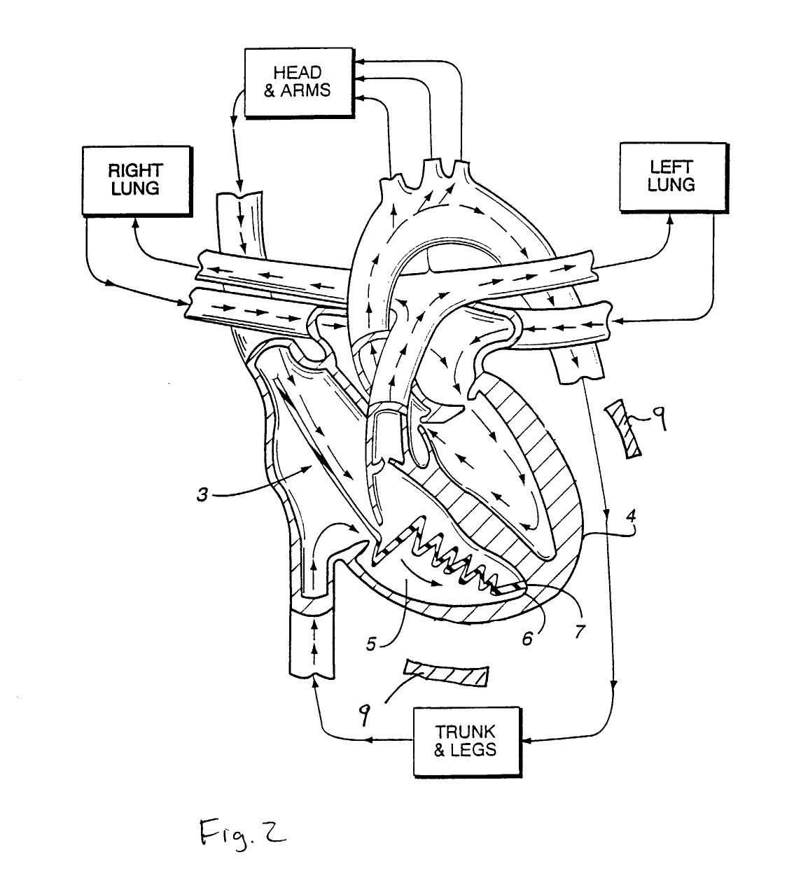

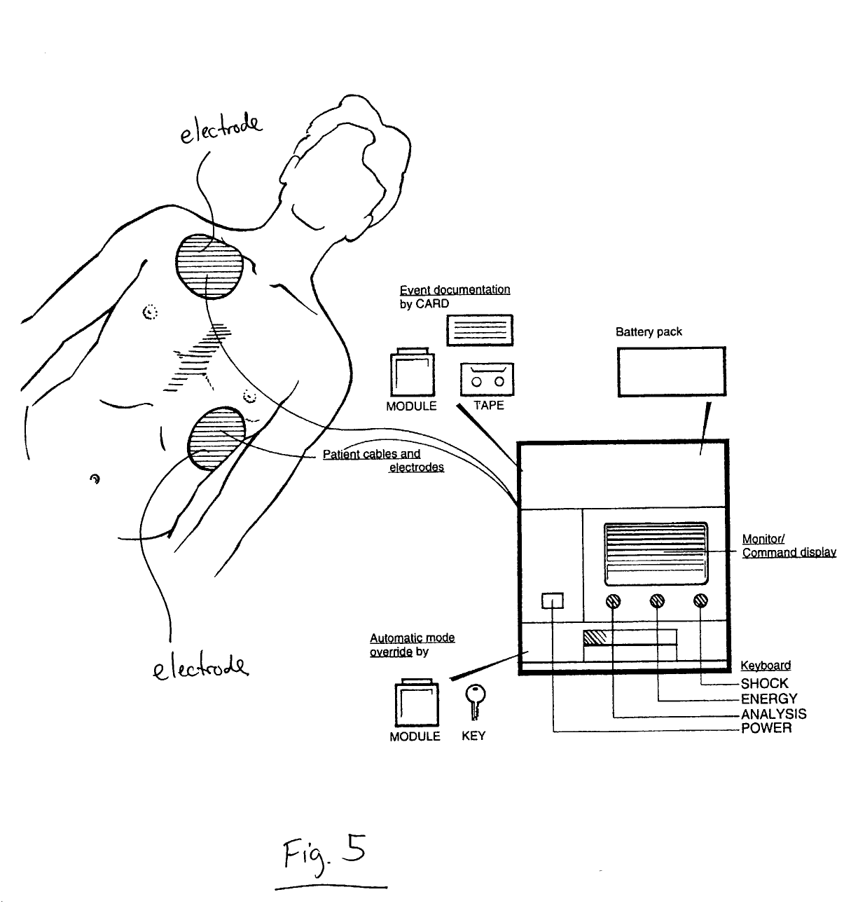

The electric shock in step B of FIG. 1 may be applied in any suitable manner. For example and without limitation, reference is made to FIG. 2 of the instant application in this regard. FIG. 2 illustrates an internal lead 3 placed in the right ventricle 5 of a patient's heart 4. The distal tip electrode 6 is located in the right ventricular apex 7. Labeled boxes in the figure illustrate the directions in which blood is pumped throughout the body by the heart. Electric energy such as shock(s) may be applied between the illustrated internal electrode and external electrode(s) 9 which may be located on the chest for example in order to induce VF in step B (which may include RF in certain circumstances as explained above). Alternatively, a plurality of external electrodes (and no internal electrode(s)) on the chest or other suitable area may be used to apply the electric energy used to induce VF in step B (e.g., see FIG. 5). Any other suitable technique may also be used in different embodiments of this invention.

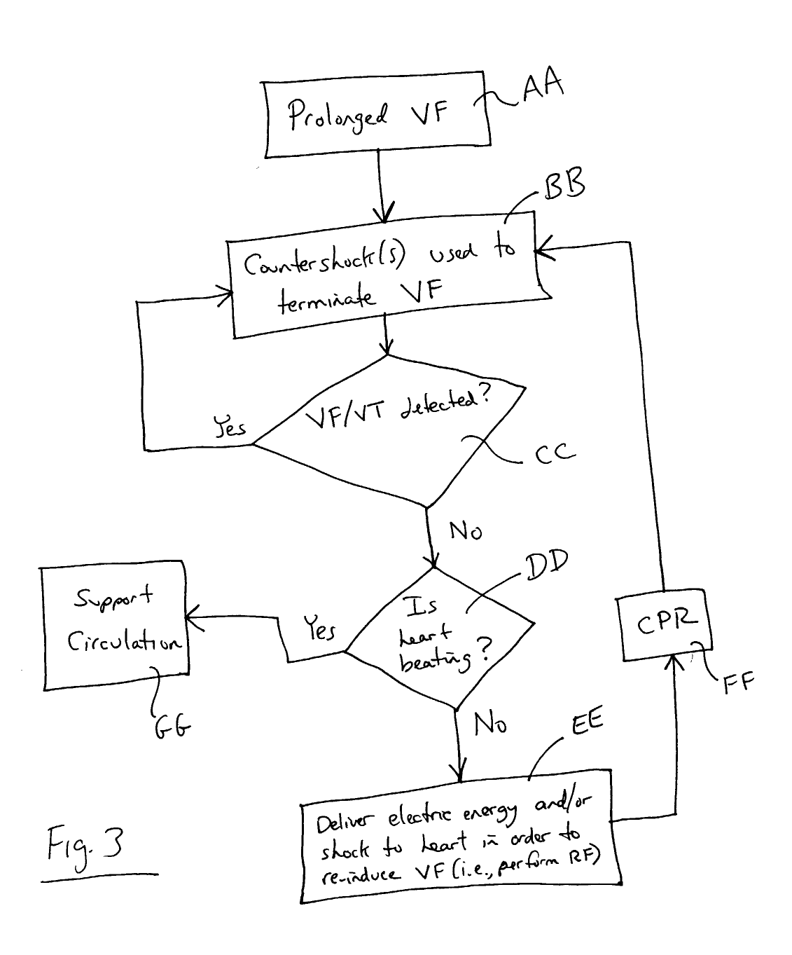

FIG. 3 is a flowchart illustrating certain steps carried out according to another embodiment of this invention. This embodiment is similar to that of FIGS. 1-2 described above, except that additional steps are provided in this embodiment. Referring to FIG. 3, a patient's heart suffers from prolonged VF (step AA). With respect to step AA, it may or may not be known whether the VF is “prolonged” at the time of treatment. Electrical shock applied via electrodes is used to terminate the VF (step BB). Thereafter, it is determined whether VF or VT are detected (step CC). If so, then countershock(s) may be continued (defibrillation is repeated). If VF and VT are not detected in step CC, then a determination is made as to whether or not the patient's heart is beating in a satisfactory manner (i.e., whether satisfactory blood flow is being generated) (step DD). If so, then no RF is performed and circulation is supported (step GG). However, if there is not satisfactory blood flow in the heart (i.e., if the answer to the step DD query is No), then an electrical shock(s) is applied to the heart in order to induce VF (step EE). It is noted that the VF in step EE may be referred to as either VF or RF in this instance since the heart previously experienced VF in step AA and the PEA and/or asystole was caused by termination of the VF in step BB. Following the re-induction of VF in step EE, CPR is performed in order to restore satisfactory blood flow (step FF), potentially followed by additional shock(s) to terminate the VF (step BB).

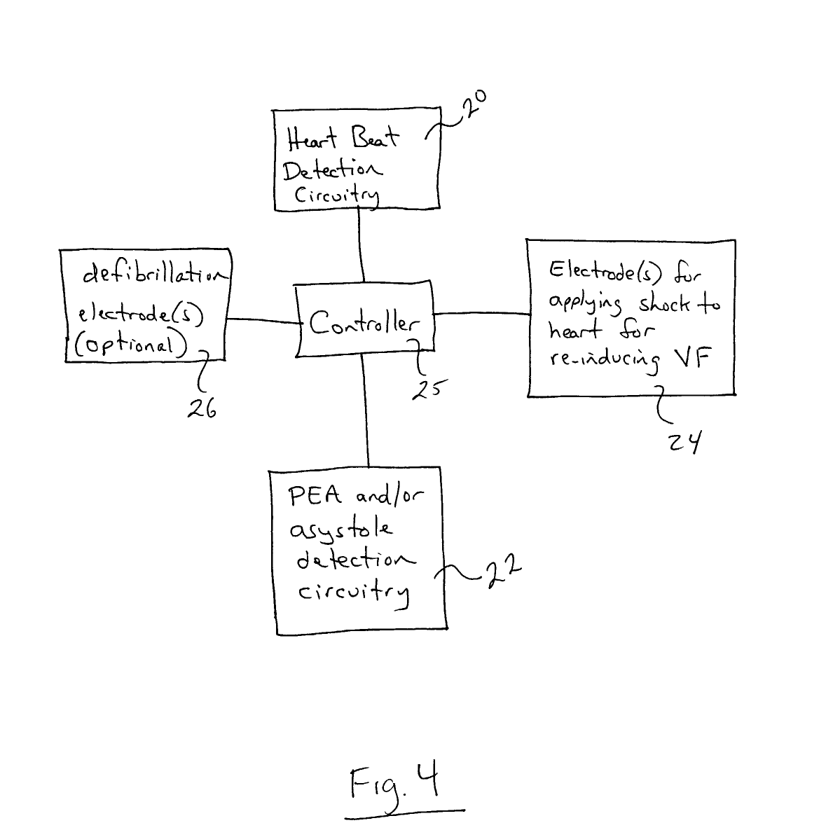

FIG. 4 is a circuit diagram of an example device for inducing VF in order to treat PEA and/or asystole according to an example embodiment of this invention. In certain example embodiments of this invention, this device may either be part of a defibrillator, or alternatively may be used alongside a defibrillator. The FIG. 4 device may monitor the heart beat via circuitry 20, and determine if ventricular fibrillation (VF) is followed by PEA or asystole via detection circuitry 22. If PEA and/or asystole is present as determined by circuitry 22, and there is a measured lack of blood flow (the lack of blood flow may be detected by the heart beat detection circuitry 20 in certain embodiments, or alternatively by other circuitry in other embodiments of this invention), then the device can deliver electrical energy to induce ventricular fibrillation (e.g., RF) via electrodes 24. The electrodes 24 used for applying shock to induce VF may or may not be the same electrodes as used to defibrillate when the device is part of a defibrillating device. The impedance measuring technique described above may be used in certain embodiments to determine if there is adequate blood flow in certain embodiments. Controller 25 is in communication with and controls and/or receives input from the aforesaid circuits. According to certain example embodiments of this invention, the FIG. 4 device may be incorporated into, or work alongside, a defibrillation device (e.g., see defibrillation electrodes 26) that is used in the treatment of cardiac arrest.

FIG. 5 is a schematic diagram of the device of FIG. 4 being used on a human patient. It can be seen that the electrodes on the chest (with no internal electrode in this embodiment) are used to induce and/or reinduce VF as discussed above in order to treat PEA or asystole.

As explained above, any suitable technique may be used for inducing VF in the heart via electrodes 24. For example, electrodes 24 may induce VF (possibly RF) in the heart by delivering a low energy electrical shock to the heart in the electrically vulnerable phase (e.g., during T-wave) in certain embodiments of this invention. Alternatively, 60 Hz may be applied to the heart via electrodes 24 in order to induce VF (e.g., the 60 Hz may be applied for a time period of from about 0.5 to 3 seconds in certain example embodiments, or possibly from 1-2 seconds in some case). In still other embodiments of this invention, direct current (DC) may be applied to the heart in a manner sufficient to induce VF. It will be appreciated that the method of inducing VF in the heart is not intended to be limiting herein, unless specifically claimed.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims. This device produces ventricular fibrillation to treat lethal cardiac rhythms. It also monitors blood flow, so that it will not induce ventricular fibrillation if the heart is beating.

1. A method of treating pulseless electrical activity (PEA) or asystole in a heart of a patient, the method comprising:
determining if the heart is in PEA or asystole; and
when it is determined that the heart is in PEA or asystole, then applying electric energy in order to induce ventricular fibrillation (VF) in the heart and thereafter performing cardiopulmonary resuscitation (CPR).

2. The method of claim 1, wherein the electric energy that is applied in order to induce VF is applied via a plurality of electrodes.

3. The method of claim 2, wherein all of the electrodes are external to the heart.

4. The method of claim 2, wherein at least one of the electrodes is external to the heart and at least another of the electrodes is internal to the heart.

5. The method of claim 1, further comprising determining whether or not the heart is beating to generate a predetermined level of blood flow before applying the electric shock in order to induce the VF, so that the electric energy is applied to induce the VF only when it is determined that the heart is not beating to generate the predetermined level of blood flow.

6. The method of claim 1, further comprising the step of detecting VF and using an electric shock to terminate the VF thereby causing the heart to be in PEA or asystole, and thereafter applying the electric energy in order to induce VF.

7. A method of treating pulseless electrical activity (PEA) or asystole in a heart of a patient, the method comprising:
determining if the heart is in PEA or asystole; and
when it is determined that the heart is in PEA or asystole, then applying electric energy in order to induce ventricular fibrillation (VF) in the heart.

8. A device for inducing ventricular fibrillation (VF) in a heart of a patient, the device comprising:
detection circuitry for determining whether the heart is in PEA or asystole; and
a plurality of electrodes for inducing VF in the heart in response to detecting at least that the heart is in PEA or asystole.

9. The device of claim 8, further comprising blood flow detection circuitry for determining whether or not the heart is beating to generate at least a predetermined level of blood flow, and wherein the VF is not induced via the plurality of electrodes unless it is determined that the heart is not beating to generate at least the predetermined level of blood flow.

10. The device of claim 8, further comprising defibrillation circuitry for causing electric shock to be applied to a heart for terminating VF.

11. A device for inducing ventricular fibrillation (VF) in a heart of a patient, the device comprising:
detection circuitry for determining whether the heart is in PEA; and
a plurality of electrodes for inducing VF in the heart in response to detecting at least that the heart is in PEA.
